(19) United States
(12) Patent Application Publication
(10) Pub. No.: US 2008/0124767 A1
(43) Pub. Date: May 29, 2008

(54) Production and Secretion of Sucrose in Photosynthetic Prokaryotes (Cyanobacteria)

(75) Inventors: David R. Nobles, Austin, TX (US); R. Malcolm Brown, Austin, TX (US)

Correspondence Address: CHALKER FLORES, LLP, 2711 LBJ FRWY, Suite 1036, DALLAS, TX, 75234, US

(73) Assignee: Board of Regents, The University of Texas at Austin, Austin, TX (US)

(21) Appl. No.: 11/866,879

(22) Filed: Oct. 3, 2007

Related U.S. Application Data
(60) Provisional application No. 60/849,363, filed on Oct. 4, 2006.

(51) Int. Cl. C12P 19/44; C12N 1/12

(52) U.S. Cl. 435/74; 435/257.1

(57) ABSTRACT

The present invention includes compositions and methods for making and producing sucrose from cyanobacteria, by growing a cyanobacterium in a growth medium; incubating the cyanobacteria in a salt containing medium under conditions that promote sucrose production; and exposing the cyanobacteria to acidic conditions, wherein the acidic conditions trigger sucrose secretion into the medium. The compositions and methods of the present invention may be used as a new global crop for the manufacture of sucrose, glucose, or fructose, CO₂ fixation, for the production of alternative sources of conventional cellulose as well as a biofuel and precursors thereof.

### CROSS-REFERENCE TO RELATED APPLICATIONS

This application claims priority to U.S. Provisional Patent Application Ser. No. 60/849,363, filed Oct. 4, 2006, the entire contents of which are incorporated herein by reference.

### TECHNICAL FIELD OF THE INVENTION

The present invention relates in general to the field of renewable fuel sources and carbon fixation, and more particularly, to the production, isolation and use of sucrose produced and harvested from cyanobacteria.

### BACKGROUND OF THE INVENTION

Without limiting the scope of the invention, its background is described in connection with feedstock for ethanol production.

Cellulose biosynthesis has a significant impact on the environment and human economy. The photosynthetic conversion of CO₂ to biomass is primarily accomplished through the creation of the cellulosic cell walls of plants and algae (Lynd et al., 2002). With approximately 10¹¹ tons of cellulose created and destroyed annually (Hess et al., 1928), this process ameliorates the adverse effects of increased production of greenhouse gasses by acting as a sink for CO₂ (Brown, 2004). Although cellulose is synthesized by bacteria, protists, and many algae, the vast majority of commercial cellulose is harvested from plants.

### SUMMARY OF THE INVENTION

More particularly, the present invention includes compositions, methods, systems and kits for producing sucrose from cyanobacteria, by growing a cyanobacterium in a growth media; incubating the cyanobacteria in a salt containing medium under conditions that promote sucrose production; and exposing the cyanobacteria to acidic conditions, wherein the acidic conditions trigger sucrose secretion into the medium. In one aspect, the method includes also includes the step of processing the sucrose into ethanol. In another aspect, the cyanobacteria are returned unharmed to growth media for continued growth and production. In another aspect, the method includes using the sucrose as a renewable feedstock for biofuel production. Generally, the cyanobacterium fixes CO₂ and thus atmospheric CO₂ using the present invention into a renewable feedstock of saccharides for, e.g., animals. In one aspect, the method creates the acidic conditions for sucrose harvesting by pumping or introducing CO₂ into the medium used for harvesting the sucrose. In one aspect, the acidic conditions are at a pH of 6 or less. The acidic condition for sucrose harvesting may include resuspending the cyanobacteria in 10 mM sodium acetate pH 5.2. In certain aspects, the sucrose secreted exceeds 1 milligram per milliliter.

Another embodiment of the present invention includes a method of fixing carbon by growing a sucrose-producing cyanobacterium in a CO₂-containing growth medium; generating sucrose with said cyanobacterium, wherein CO₂ is fixed into sucrose at a level higher than an unmodified cyanobacterium; and calculating the amount of CO₂ fixed into the sucrose to equate to one or more carbon credit units. For example, at least one other carbon may be fixed into sucrose and the at least one other carbon's is equated to carbon credit units that is included in the calculation. The method may further include the step of processing the sucrose into ethanol, e.g., as a renewable feedstock for biofuel production. Generally, the cyanobacterium fixes CO₂ and thus atmospheric CO₂ using the present invention into a renewable feedstock of saccharides for, e.g., animals. Importantly, it has been found that the cyanobacteria of the present invention produce sucrose, but also secrete the sucrose into the medium under certain conditions.

In one aspect, the method creates the acidic conditions for sucrose harvesting by pumping or introducing CO₂ into the medium used for harvesting the sucrose. In one aspect, the acidic conditions are at a pH of 6 or less. The acidic condition for sucrose harvesting may include resuspending the cyanobacteria in 10 mM sodium acetate pH 5.2. In certain aspects, the sucrose secreted exceeds 1 milligram per milliliter.

Another embodiment of the present invention includes an isolated cyanobacterium comprising a portion of an exogenous bacterial cellulose operon sufficient to express bacterial saccharides, whereby the cyanobacterium is capable of producing secretable monosaccharides, disaccharides, oligosaccharides or polysaccharides that comprise sucrose.

A vector for expression of a portion of the cellulose operon sufficient to express bacterial cellulose operon that includes a microbial cellulose operon, e.g., the acsAB gene operon, under the control of a promoter that expresses the genes in the operon in cyanobacteria. The skilled artisan will recognize that the vector may combine portions of the operons of bacterial, algal, fungal and plant cellulose operons to maximize production and/or change the characteristics of the cellulose and may be transfer and/or expression vector.

The system for the manufacture of bacterial cellulose may further include growing an exogenous cellulose expressing cyanobacterium adapted for growth in a hypersaline environment, such that the cyanobacterium does not grow in fresh water or the salinity of sea water. The growth of the cyanobacteria in a hypersaline environment may be used as way to limit the potential for unplanned growth of the cyanobacteria outside controlled areas. In one example, the sucrose secreting cyanobacteria of the present invention may be grown in brine ponds obtained from subterranean formation, such a gas and oil fields. In another example, the secreted sucrose is processed into concentrated molasses or dry sucrose crystals, pharmaceuticals, vaccines, vitamins, industrial chemicals, proteins, pigments, fatty acids and their derivatives (such as polyhydroxybutyrate), acylglycerols (as precursors for biodiesel), and other secondary metabolites. Examples of cyanobacteria for use with the system include those that are photosynthetic, nitrogen-fixing, capable of growing in brine, facultative heterotrophs, chemoautotrophic, and combinations thereof.

### BRIEF DESCRIPTION OF THE DRAWINGS

For a more complete understanding of the features and advantages of the present invention, reference is now made to the detailed description of the invention along with the accompanying figures and in which:

FIG. 1 shows a diagram of a production plant that may be used to produce, isolate and process the saccharides produced using the present invention.

FIG. 2 shows photobioreactor design for in situ harvest of cyanobacterial saccharides.

FIG. 3 is a side view of a photobioreactor complex design for in situ harvest of cyanobacterial saccharides.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “continuous method” or “continuous feed method” refer to a fermentation method that includes continuous nutrient feed, substrate feed, cell production in the bioreactor, cell removal (or purge) from the bioreactor, and product removal. Such continuous feeds, removals or cell production may occur in the same or in different streams. A continuous process results in the achievement of a steady state within the bioreactor. As used herein, the term “steady state” refers to a system and process in which all of these measurable variables (i.e., feed rates, substrate and nutrient concentrations maintained in the bioreactor, cell concentration in the bioreactor and cell removal from the bioreactor, product removal from the bioreactor, as well as conditional variables such as temperatures and pressures) are relatively constant over time.

As used herein, the terms “photobioreactor,” “photoreactor,” or “cyanobioreactor,” include a fermentation device in the form of ponds, trenches, pools, grids, dishes or other vessels whether natural or man-made suitable for inoculating the cyanobacteria of the present invention and providing to one or more of the following: sunlight, artificial light, salt, water, CO₂, H₂O, growth media, stirring and/or pumps, gravity or force fed movement of the growth media. The product of the photobioreactor will be referred to herein as the “photobiomass”. The “photobiomass” includes the cyanobacteria, secreted materials and mass formed into, e.g., cellulose or value added products whether intra or extracellular.

As used herein, the terms “bioreactor,” “reactor,” or “fermentation bioreactor,” include a fermentation device that includes of one or more vessels and/or towers or piping arrangement, which includes the Continuous Stirred Tank Reactor (CSTR), Immobilized Cell Reactor (ICR), Trickle Bed Reactor (TBR), Bubble Column, Gas lift Fermenter, Static Mixer, or other device suitable for gas-liquid contact. A fermentation bioreactor for use with the present invention includes a growth reactor which feeds the fermentation broth to a second fermentation bioreactor, in which most products, e.g., alcohols or furans are produced. In some cases, the gaseous byproduct of fermentation, e.g., CO₂, can be pumped back into the photobioreactor to recycle the gas and promote the formation of saccharides by photosynthesis. To the extent that heat is generated during the process of recovering the products of the fermentation, etc., the heat can also be used to promote cyanobacterial cell growth and production of saccharides.

As used herein, the term “nutrient medium” refers to conventional cyanobacterial growth media that includes sufficient vitamins, minerals and carbon sources to permit growth, photosynthesis and secretion of the saccharides, e.g. sucrose, by the cyanobacteria of the present invention. Components of a variety of nutrient media suitable to the use of this invention are known and reported in e.g., Cyanobacteria, Volume 167: (Methods in Enzymology) (Hardcover), by John N. Abelson Melvin I. Simon and Alexander N. Glazer (Editors), Academic Press, New York (1988).

As used herein, the term “cell concentration” refers to the dry weight of cyanobacteria per liter of sample. Cell concentration is measured directly or by calibration to a correlation with optical density.

As used herein, the term “saccharide production” refers to the amount of mono-, di-, oligo or polysaccharides produced by the modified-cyanobacteria of the present invention that produce saccharides by fixing carbon such as CO₂ by photosynthesis into the saccharides. One distinct advantage of the present invention is that the cyanobacteria do not produce lignin along with the production of the cellulose and other saccharides that can be used a feed-stock for fermentation and other bioreactors that convert the saccharides into, e.g., ethanol or other synfuels.

In operation, the present invention may use any of a variety of known fermentation process steps, compositions and methods for converting the saccharides into useful products, e.g., lignin-free cellulose, alcohols, furans and the like. One non-limiting example of a process for producing ethanol by fermentation is a process that permits the simultaneous saccharification and fermentation step by placing the saccharide source at a temperature of above 34° C. in the presence of a glucoamylase and a thermo-tolerant yeast.

In this example, the following main process stages may be included saccharification (if necessary), fermentation and distillation. One particular advantage of the present invention is that it eliminates a variety of processing steps, including, milling, bulk-fiber separations, recovery or treatments for the control or elimination of lignin, water removal, distillation and burning of unwanted byproducts. Any of the process steps of alcohol production may be performed batchwise, as part of a continuous flow process or combinations thereof.

Saccharification. To produce mono- and di-saccharides from the lignin-free cellulose of the present invention the cellulose can be metabolized by cellulases that provide the yeast with simple saccharides. This “saccharification” steps include the chemical or enzymatic hydrolysis of long-chain oligo and polysaccharides by enzymes such as cellulase, glucoamylases, alpha-glucosidase, alkaline, acid and/or thermophilic alpha-amylases and if necessary phytases.

Depending on the length of the polysaccharides, enzymatic activity, amount of enzyme and the conditions for saccharification, this step may last up to 72 hours. Depending on the feedstock, the skilled artisan will recognize that saccharification and fermentation may be combined in a simultaneous saccharification and fermentation step.

Fermentation. Any of a wide-variety of known microorganism may be used for the fermentation, fungal or bacterial. For example, yeast may be added to the feedstock and the fermentation is ongoing until the desired amount of ethanol is produced; this may, e.g., be for 24-96 hours, such as 35-60 hours. The temperature and pH during fermentation is at a temperature and pH suitable for the microorganism in question, such as, e.g., in the range about 32-38° C., e.g. about 34° C., above 34° C., at least 34.5° C., or even at least 35° C., and at a pH in the range of, e.g., about pH 3-6, or even about pH 4-5. The skilled artisan will recognize that certain buffers may be added to the fermentation reaction to control the pH and that the pH will vary over time.

The use of a feed stock that includes monosaccharides, in addition to the use of thermostable acid alpha-amylases or a thermostable maltogenic acid alpha-amylases and invertases in the saccharification step may make it possible to improve the fermentation step. When using a feedstock that includes large amounts of monosaccharides such as glucose and sucrose, for the production of ethanol it may be possible to reduce or eliminate the need for the addition of glucoamylases in the fermentation step or prior to the fermentation step.

Distillation. To complete the manufacture of final products from the saccharides made by the cyanobacterial fixation of CO₂ of the present invention, the invention may also include recovering the alcohol (e.g., ethanol). In this step, the alcohol may be separated from the fermented material and purified with a purity of up to e.g. about 96 vol. % ethanol can be obtained by the process of the invention.

Several specific enzymes and methods may be used to improve the recovery of energy containing molecules from the present invention. The enzymes improve the saccharification and fermentation steps by selecting their most efficient activity as part of the processing of the products of the saccharide producing modified cyanobacteria of the present invention.

In one example, a thermo tolerant cellulase may be introduced into the reactor to convert cellulose produced by the cyanobacteria of the present invention into monosaccharides, which will mostly be glucose. Examples of thermophilic cellulases are known in the art as taught in, e.g., U.S. Patent Application No 20030104522 filed by Ding, et al. that teach a thermal tolerant cellulase from Acidothermus cellulolyticus. Yet another example is taught by U.S. Patent Application No. 20020102699, filed by Wicher, et al., which teaches variant thermostable cellulases, nucleic acids encoding the variants and methods for producing the variants obtained from Rhodothermus marinus. The relevant portions of each are incorporated herein by reference.

Acid cellulase may be obtained commercially from manufacturers such as Ideal Chemical Supply Company, Memphis Term., USA; Americos Industries Inc., Gujarat, India; or Rakuto Kasei House, Yokneam, Israel. For example, the acid cellulase may be provided in dry, liquid or high-active abrasive form, as is commonly used in the denim acid washing industry using techniques known to the skilled artisan. For example, Americos Cellscos 450 AP is a highly concentrated acid cellulase enzyme produced using genetically modified strains of Trichoderma reesii. Typically, the acid cellulases function in a pH range or 4.5-5.5.

Microorganisms used for fermentation. One example of a microorganism for use with the present invention is a thermo-tolerant yeast, e.g., a yeast that when fermenting at 35° C. maintains at least 90% of the ethanol yields and 90% of the ethanol productivity during the first 70 hours of fermentation, as compared to when fermenting at 32° C. under otherwise similar conditions. One example of a thermotolerant yeast is a yeast that is capable of producing at least 15% V/V alcohol from a corn mash comprising 34.5% (w/v) solids at 35° C. One such thermo-tolerant yeast is Red Star®/Lesaffre Ethanol Red (commercially available from Red Star®/Lesaffre, USA, Product No. 42138). The ethanol obtained using any known method for fermenting saccharides (mono, di-, oligo or poly) may be used as, e.g., fuel ethanol, drinking ethanol, potable neutral spirits, industrial ethanol or even fuel additives.

Examples of ethanol fermentation from sugars are well-known in the art as taught by, e.g., U.S. Pat. No. 4,224,410 to Pemberton, et al. for a method for ethanol fermentation in which fermentation of glucose and simultaneous-saccharification fermentation of cellulose using cellulose and a yeast are improved by utilization of the yeast Candida brassicae, ATCC 32196; U.S. Pat. No. 4,310,629 to Muller, et al., that teaches a continuous fermentation process for producing ethanol in which continuous fermentation of sugar to ethanol in a series of fermentation vessels featuring yeast recycle which is independent of the conditions of fermentation occurring in each vessel is taught; U.S. Pat. No. 4,560,659 to Asturias for ethanol production from fermentation of sugar cane that uses a process for fermentation of sucrose wherein sucrose is extracted from sugar cane, and subjected to stoichiometric conversion into ethanol by yeast; and U.S. Pat. No. 4,840,902 to Lawford for a continuous process for ethanol production by bacterial fermentation using pH control in which a continuous process for the production of ethanol by fermentation of an organism of the genus Zymomonas spp. is provided. The method of Lawford is carried out by cultivating the organism under substantially steady state, anaerobic conditions and under conditions in which ethanol production is substantially uncoupled from cell growth by controlling pH in the fermentation medium between a pH of about 3.8 and a pH less than 4.5; and K A Jacques, T P Lyons & D R Kelsall (Eds) (2003), The Alcohol Textbook; 4^{TH} Edition, Nottingham Press; 2003. The relevant portions of each of which are incorporated herein by reference.

One of ordinary skill in the art would recognize that the quantity of yeast to be contacted with the photobiomass will depend on the quantity of the photobiomass, the secreted portions of the photobiomass and the rate of fermentation desired. The yeasts used are typically brewers' yeasts. Examples of yeast capable of fermenting the photobiomass include, but are not limited to, Saccharomyces cerevisiae and Saccharomyces uvarum. Besides yeast, genetically altered bacteria know to those of skill in the art to be useful for fermentation can also be used. The fermenting of the phototbiomass is conducted under standard fermenting conditions.

Separating of the ethanol from the fermentation can be achieved by any known method (e.g. distillation). The separation can be performed on either or both the liquid and solid portions of the fermentation solution (e.g., distilling the solid and liquid portions), or the separation can just be performed on the liquid portion of the fermentation solution (e.g., the solid portion is removed prior to distillation). Ethanol isolation can be performed by a batch or continuous process. The separated ethanol, which will generally not be fuel-grade, can be concentrated to fuel grade (e.g., at least 95% ethanol by volume) via additional distillation or other methods known to those of skill in the art (e.g., a second distillation).

The level of ethanol present in the fermentation solution can negatively affect the yeast/bacteria. For example, if 17% by volume or more ethanol is present, then it will likely begin causing the yeast/bacteria to die. As such, ethanol can be separated from the fermentation solution as the ethanol levels (e.g., 12, 13, 14, 15, 16, to 17% by volume (ethanol to water)) that may kill the yeast or bacteria are reached. Ethanol levels can be determined using methods known to those of ordinary skill in the art.

The fermentation reaction can be run multiple times on the photobiomass or portions thereof. For example, once the level of ethanol in the initial fermentation reactor reaches 12-17% by volume, the entire liquid portion of the fermentation solution can be separated from the biomass to isolate the ethanol (e.g., distillation). The “once-fermented” photobiomass can then be contacted with water, additional enzymes and yeast/bacteria for additional fermentations, until the yield of ethanol is undesirably low. Factors that the skilled artisan will use to determine the number of fermentations include: the amount of photobiomass remaining in the vessel; the amount of carbohydrate remaining, the type of yeast or bacteria, the temperature, pH, salt concentration of the media and overall ethanol yield. If any carbohydrates remain, then the remaining photobiomass is removed from the vessel.

Generally, it is desirable to isolate or harvest the yeast/bacteria from the fermentation reaction for recycling. The method of harvesting will depend upon the type of yeast/bacteria. If the yeast/bacteria are top-fermenting, they can be skimmed off the fermentation solution. If the yeast/bacteria are bottom-fermenting, they can be removed from the bottom of the tank.

Often, a by-product of fermentation is carbon dioxide, which is readily recycled into the photobioreactor for fixation into additional saccharides. During the fermentation process, it is expected that about one-half of the decomposed starch will be discharged as carbon dioxide. This carbon dioxide can be collected by methods known to those of skill in the art (e.g., a floating roof type gas holder) and is supplied back into the photobioreactor pool or pools. In colder climates, the heat that may accompany the carbon dioxide will help in the growth of the cyanobacterial pools.

One advantage of the present invention is that it provides a novel CO₂ fixation method for the recycling of environmental greenhouse gases. If successful on a large scale, this new global cellulose crop will sequester CO₂ from the air, thus reducing the potential greenhouse gas responsible for global warming. Another benefit of the present invention is the cyanobacteria can be grown on non-arable land, thus freeing the land to allow regeneration of forests and use of cropland for other needs.

The data shown in Table 1 demonstrate that cyanobacterial sucrose can yield approximately 625 gallons of ethanol per acre foot per year by direct fermentation with Zymomonas mobilis. Currently, starch from corn yields 400 gallons of ethanol per acre. Direct fermentation of sucrose will yield cost benefits over corn starch which must be digested with amylase prior to fermentation.

Despite its superior quality, the use of microbial cellulose as a primary constituent for large scale use in common applications such as the production of construction materials, paper, or cardboard has not been economically feasible. The root cause for the expense of microbial cellulose production is the heterotrophic nature of A. xylinum. Bacterial cultures must be supplied with glucose, sucrose, fructose, glycerol, or other carbon sources produced by the cultivation of plants. Increased distance from the primary energy source is inherently less efficient and inevitably leads to increased cost of production when compared with phototrophic sources. Therefore, while the unique properties of A. xylinum cellulose make it indispensable for a number of value added products, it is not well suited for the more general applications that constitute the vast majority of cellulose utilization (Brown, 2004; White and Brown, 1989), e.g., to replace the use of forests for the production of paper and to provide substrates for the production of biofuels based on ethanol using photosynthesis as the source of energy for CO₂ fixation. As such, the present invention provides compositions and methods for the manufacture of a new global crop that may be used for energy production and removal of the greenhouse gas CO₂ using an environmentally acceptable natural process that requires little or no energy input for manufacture.

Unlike A. xylinum, cyanobacteria require no fixed carbon source for growth. Additionally, many cyanobacteria are capable of nitrogen fixation, which would eliminate the need for fertilizers necessary for cellulose crops like cotton. In addition, many cyanobacteria are halophilic, that is, they can grow in a range of brackish to hypersaline environments. This feature, in combination with N-fixation, will allow non-arable, sun-drenched areas of the planet to provide the extensive surface areas for the growth and harvest of cellulose made using the compositions and methods of the present invention on a global scale.

Cyanobacterial cellulose can be used in diverse applications where a combination of products is simultaneously made from photosynthesis. Value added products may include pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. These products may be the result of natural cyanobacterial metabolic processes or be induced through genetic engineering. The present invention permits large scale production of cellulose, proteins and other products that may be grown and harvested. In fact, wide application of the cells themselves for glucose and cellulose is encompassed by the present invention. The cellulose producing cyanobacteria of the present invention may be utilized for energy recycling and recovery, that is, the cells may be dried and burned to power downstream processes in a manner similar to the use of bagasse in the sugar cane industries

### EXAMPLE 1

Culture Conditions. Synechococcus leopoliensis UTCC 100 (also known as Synechococcus elongatus PCC 7942) was maintained at 24° C. with 12 hour light/dark cycles in BGll (Allen, 1968) or BG11 supplemented with 1% w/v NaCl. Solid media was prepared with 1.5% agar as previously described (Golden et al, 1988). 50 ml liquid cultures were maintained on a rotary shaker in 250 ml Erlenmeyer flasks. Cell concentrations of cultures were determined by measuring their optical density at 750 nm (OD₇₅₀).

Determination of Sucrose Concentrations. Preparation of Cultures. 50 ml liquid cultures were initially inoculated from agar plates. The entire cell mass from each 50 ml culture was recycled after each harvest. Cells were routinely allowed to grow 7-12 days before sucrose induction. A shorter 2-3 day growth period was also implemented as a method for increasing sucrose production. After the appropriate growth period, the OD₇₅₀ was recorded. Cells were collected by centrifugation (10 min, RT, 1,744×g) in an IEC clinical centrifuge. The supernatants were discarded and wet weights of the cell pellets were recorded. Cell pellets were resuspended in 50 ml BGll supplemented with 2% w/v NaCl then allowed to grow overnight under the above culture conditions. After recording the OD₇₅₀, cells were collected by centrifugation as above and the wet weight of the cell pellet was recorded. For induction of sucrose release, pellets were resuspended in 1 ml of 10 mM Sodium Acetate, pH 5.2. 500 ul aliquots of the cell suspension were transferred to 1.5 ml eppendorf tubes. The tubes were incubated 2 hours on a rotisserie at 30° C. with constant illumination.

Sucrose Assays. After incubation, cells were pelleted by centrifugation (5 min, RT, 14,000 rpm) in a microcentrifuge. The supernatant was carefully pipetted off the cell pellet and retained for the sucrose assay. Sucrose concentration was determined by digestion with invertase (Sigma S1299) followed by the hexokinase-glucose 6-phosphate dehydrogenase enzymatic assay (Sigma G3293). Assays were performed with 50 ul of supernatant per reaction following the manufacturer's instructions.

Tables 1 and 2 demonstrate significant sucrose production by S. leopoliensis UTCC 100. Assuming lossless scale-up, these preliminary results predict theoretical yields of approximately 5 tons acre ft⁻¹ year⁻¹ for routine collection and 8 tons acre ft⁻¹ year⁻¹ for serial harvests. Although these amounts fall short of the sucrose production levels of sugarcane (9 tons acre⁻¹ year⁻¹), the ease of sucrose harvest, use of brackish or briny water, and location neutrality of cyanobacteria offer competitive advantages over land-based crops that may offset deficits in production levels.

TABLE 1
Sucrose production levels for routine collection method.

| | Wet | Sucrose | mg Sucrose | mg Sucrose |
|---|---|---|---|---|
| OD₇₅₀ | Weight (g) | (mg/ml) | g̅ W̅e̅t̅ W̅e̅i̅g̅h̅t̅ | liter |
| 1.46 +/− | 0.024 +/− | 2.28 +/− | 8.60 +/− | 57.01 +/− |
| 0.06 | 0.05 | 0.52 | 1.91 | 13.00 |

TABLE 2
Serial sucrose harvests conducted over one week.

| | | Wet | Sucrose | mg Sucrose | mg Sucrose |
|---|---|---|---|---|---|
| | OD₇₅₀ | Weight (g) | (mg/ml) | g̅ W̅e̅t̅ W̅e̅i̅g̅h̅t̅ | liter |
| Day 1 | 1.4 | 0.17 | 2.01 | 11.82 | 50.25 |
| Day 4 | 1.2 | 0.20 | 1.33 | 6.65 | 33.25 |
| Day 7 | 1.2 | 0.17 | 1.12 | 6.59 | 28.00 |

The production of sucrose in response to salt stress has previously been demonstrated in Synechococcus elongatus PCC 7942 (Nectarios and Papageorgiou, 2000). However, to our knowledge, the secretion of sucrose has not been observed prior to this research. Since cells appear to be unharmed by the process, it seems likely that the release of sucrose into the external milieu is facilitated by a specific sucrose secretion mechanism rather than release due to cell membrane instability. Interestingly, an acidic environment seems to be required to liberate significant amounts of sucrose. If glass distilled H₂O is used in place of acidic buffer for induction, the yield of sucrose is only about 1/10 that observed when buffer is used (data not shown). The possibility of an active sucrose secretion system suggests a possible avenue for increasing production levels. Additional possibilities for improved yields may come from engineering of components of starch and sucrose metabolism pathways.

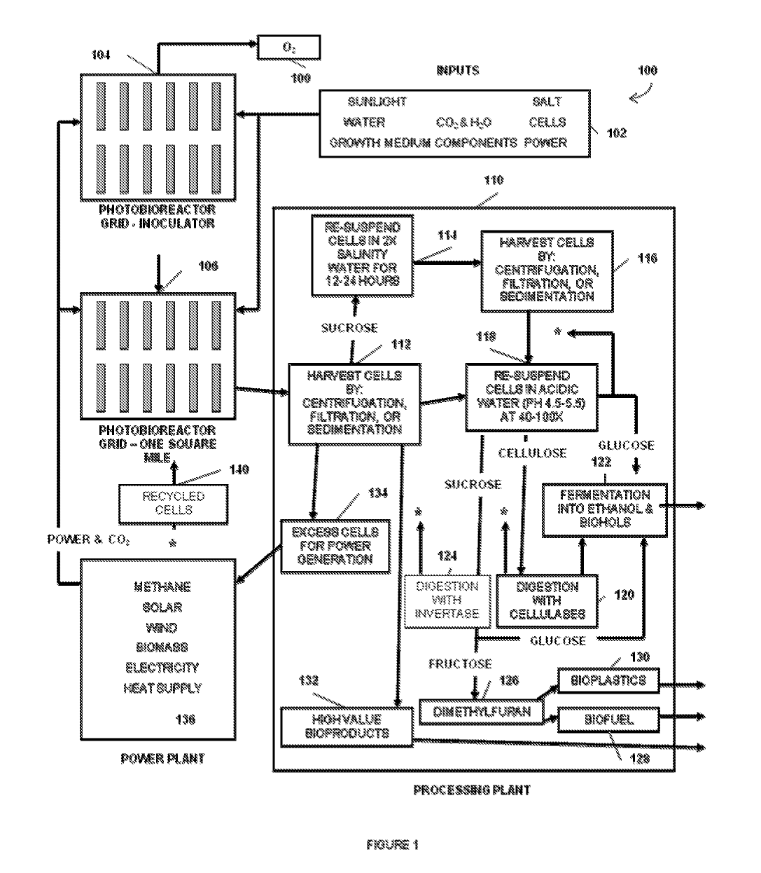

FIG. 1 shows one example of a photobioreactor system 100 of the present invention. First, inputs 102 for the photobioreactor system may include: sunlight, artificial light, salt, water, CO₂ modified-cyanobacterial cells of the present invention, growth medium components and if necessary a source of power to move the components (e.g., pumps or gravity). Next, the inputs 102 and inoculated into a photobioreactor grid 104 that is used to grow the modified-cyanobacteria in size and number, to test for saccharide production and to reach a sufficiently high enough concentration to inoculate the operating photobioreactor 106. The photobioreactor 106 may be a pool or pool(s), trench or other vessel, indoor or outdoor that is used to grow and harvest a sufficient volume of photobiomass for subsequent processing in, e.g., processing plant 110. In one example, the photobioreactor 106 may be a grid of pools of one square mile (or larger) that may be used in parallel or in series to produce the photobiomass. Depending on the geographical location of the photobioreactor 106, the water may be saltwater or brine obtained from a sea that is gravity fed into the pools. Gravity or pumping may be used, however, gravity has the advantage that it does not require additional energy to move the photobiomass from pool to pool and even into the processing plant. In fact, in certain embodiments the entire system may be gravity fed with the final products gravity fed into underground rivers that return to the sea or ocean.

The processing plant 110 includes a cell harvested 112, which may allows the isolation of the photobiomass by, e.g., centrifugation, filtration, sedimentation, creaming or any other method for separating the photobiomass, the modified-cyanobacterial cells and the liquid. For the isolation of sucrose, the cells may be resuspended in medium with an increased salinity 114 (e.g., 2× the salinity) followed by a second harvesting step 116. The twice-harvested cells are then resuspended under acidic conditions (e.g., pH 4.5-5.5) at 40 to 100× the concentration and the sucrose is secreted by the modified-cyanobacteria. If glucose is preferred, the once harvested cells are resuspended under acidic conditions 118 and glucose is secreted. In addition, whether sucrose or glucose is secreted, cellulose is also harvested from the modified-cyanobacteria, which may be further digested by cellulases 120. Glucose and digested cellulose can then be fermented into ethanol or other alcohols.

Sucrose can be converted into glucose and fructose, fructose can be made into dimethylfuran. If sucrose is secreted and obtained, then the sucrose can be converted into dimethylfuran and glucose by an invertase enzyme 124. The methylfuran 12 can then be used for bioplastic 130 or biofuel 128 production. Glucose that is obtained after the invertase reaction 124 may then be redirected back into the fermentation reactions.

In addition to the production of ethanol, bioplastics and other biofuels, the harvested cells can be used for the production of other high value bioproducts, e.g., by further modifying the microbial cellulose-producing cyanobacteria to make other bioproducts, e.g., pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. After each of these steps, the modified-cyanobacteria can then be recycled into the photobioreactors for additional carbon fixation. Furthermore, the products of the processing plant 110 can also be combined with other power sources, e.g., solar, methane, wind, etc., to generate electricity and heat (in addition to recycling any CO₂ released in the processing plant 110), to power the inoculation pool 104 and the photobioreactor 106.

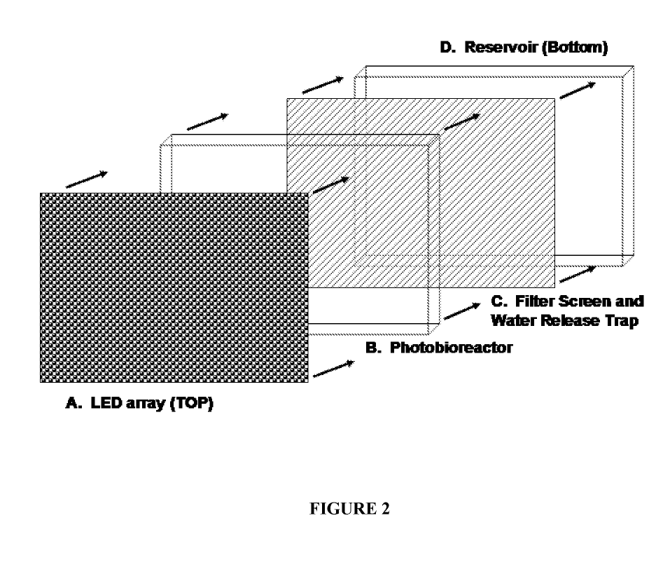

FIG. 2 shows a photobioreactor design for in situ harvest of cyanobacterial saccharides. The photobioreactor complex can be located indoors or underground. Part A An LED array powered by photovoltaic cells, provides mono or polychromatic light at a pulsed frequencies corresponding to the rate limiting steps of photosynthesis for maximized photosynthetic productivity. Part B is a transparent photobioreactor acting as a growth vessel for cyanobacterial cells. The horizontal orientation of the photobioreactor allows for efficient separation of cells from culture medium by use of gravity and air pressure. Part C is a filter screen combined with a liquid release trap will separate cells from the culture medium. The filter screen will have pore sizes capable of retaining cyanobacterial cells while allowing culture medium to flow into the reservoir. The transfer will be facilitated by gravity and air pressure generated by closing the gas outlet of the photobioreactor. The reservoir, located beneath the photobioreactor, will act to retain culture medium during harvest of saccharides. After harvest, culture medium will be returned to the photobioreactor from the reservoir via pump.

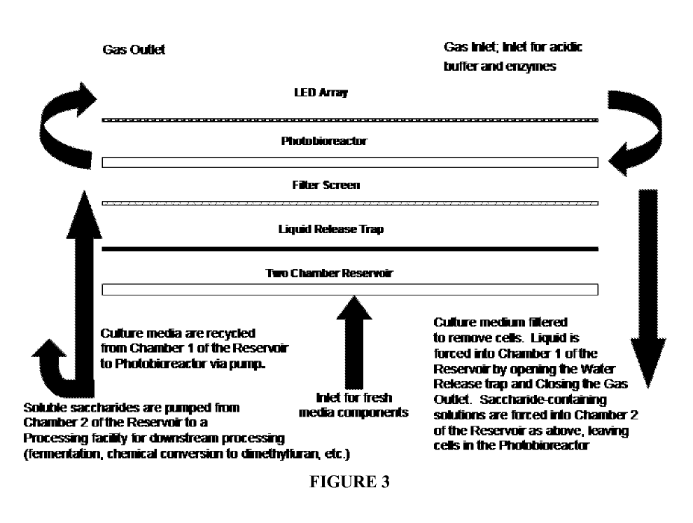

FIG. 3 shows the operation of a photobioreactor complex design for in situ harvest of cyanobacterial saccharides. The LED array, located on top of the photobioreactor complex will supply pulsed mono or polychromatic light for maximum photosynthetic conversion efficiency. Air flow (CO₂, N₂, or ambient air) delivered by the gas inlet during growth periods will serve to deliver carbon and/or nitrogen sources for fixation and created turbulence for maintaining cell suspension. A gas outlet will facilitate the release of waste gasses (O₂ and H₂) that are potentially detrimental to the cyanobacterial growth and relieve excess air pressure from the system during growth phases. Removal of culture media for harvesting of saccharides will be facilitated by the opening of the liquid release trap coupled with closing the gas outlet. The increase in air pressure, combined with gravity, will force the culture medium through the filter which will retain cyanobacterial cells. Cyanobacterial cells can then be resuspended in specific buffer or media designed for cellulose digestion or the direct secretion of saccharides. The saccharide-containing solutions will be drained to chamber 2 of the liquid release trap by the same method described for growth media above. Soluble saccharides will be pumped from chamber 2 of the reservoir to central processing units for downstream conversion processes (e.g., fermentation, chemical conversion to dimethylfuran, etc.). Cells will be resuspended by closing the water release trap and pumping culture medium which has been recombined with fresh media components (e.g., nitrates, phosphates, etc.) from chamber 1 of the reservoir.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Allen M. (1968). Simple conditions for growth of unicellular blue green algae on plates. J Phycol 4: 1-4.
Golden S S, Brusslan J, Haselkorn R. (1988). Mutagenesis of cyanobacteria by classical and gene-transfer-based methods. In Packer L and Glazer A N (eds) Methods in Enzymology ed. Vol. 167 pp 714-727. Academic Press, Inc. New York.
Nobles D R, Romanovicz D K, Brown R M Jr. (2001). Cellulose in cyanobacteria. Origin of vascular plant cellulose synthase? Plant Physiol. 127(2):529-42.
Roberts E. (1991). Biosynthesis of Cellulose II and Related Carbohydrates PhD thesis. The University of Texas at Austin, Austin.
Roelofsen P A. (1959). The plant cell wall constituents. In: The Plant Cell Wall. Gebrüder Borntraeger (ed). Felengraff and Co. Berlin. pp 1-33.
Sakamoto T and Bryant D A. (1998). Growth at low temperature causes nitrogen limitation in the cyanobacterium Synechococcus sp. PCC 7002. Arch Microbiol. 169: 10-19
Saxena I M, Kudlicka K, Okuda K, Brown R M Jr. (1994) Characterization of genes in the cellulose synthesizing operon (acs operon) of Acetobacter xylinum: implications for cellulose crystallization. J Bacteriol 176: 5735-5752.
Stevens S E Jr, Patterson C O P, Myers J. (1973). The production of hydrogen peroxide by blue-green algae: a survey. J. Phycol. 9:427-430.
Tel-Or E, Spath S, Packer L, and Mehlhorn R J. (1986). Carbon-13 NMR Studies of Salt Shock-Induced Carbohydrate Turnover in the Marine Cyanobacterium Agmenellum quadruplicatum. Plant Physiol. 82: 646-652.
Updegraff D M. (1969). Semimicro determination of cellulose in biological material. Anal Biochem. 32(3):420-424.

What is claimed is:

1. A method of producing sucrose from cyanobacteria, comprising:
growing the cyanobacteria in a growth media;
incubating the cyanobacteria in a salt containing medium under conditions that promote sucrose production; and
exposing the cyanobacteria to acidic conditions, wherein the acidic conditions trigger sucrose secretion into the medium.

2. The method of claim 1, further comprising the step of processing the sucrose into ethanol.

3. The method of claim 1, wherein the sucrose is used as a renewable feedstock for biofuel production.

4. The method of claim 1, wherein the cyanobacterium fixes one or more of the following: N₂, CO₂ and thus atmospheric CO₂.

5. The method of claim 1, wherein the sucrose is used as a renewable feedstock for animals.

6. The method of claim 1, wherein the acidic conditions are created by pumping CO₂ into the medium.

7. The method of claim 1, wherein the acidic conditions comprise a pH of 6 or less.

8. The method of claim 1, wherein the acidic condition comprises resuspending the cyanobacteria in 10 mM sodium acetate pH 5.2.

9. The method of claim 1, wherein the sucrose secreted exceeds 1 milligram per milliliter.

10. A method of fixing carbon comprising:
growing a sucrose-producing cyanobacterium in a CO₂-containing growth medium;
generating sucrose with said cyanobacterium, wherein CO₂ is fixed into sucrose; and
calculating the amount of CO₂ fixed into the sucrose to equate to one or more carbon credit units.

11. The method of claim 10, wherein at least one other carbon is fixed into sucrose and the at least one other carbon's is equated to carbon credit units that is included in the calculation.

12. The method of claim 10, further comprising the step of processing the sucrose into ethanol.

13. The method of claim 10, wherein the sucrose is used as a renewable feedstock for biofuel production.

14. The method of claim 10, wherein the cyanobacterium fixes CO₂ and the amount of CO₂ fixed is converted into one or more carbon credits.

15. The method of claim 10, wherein the sucrose is used as a renewable feedstock for animals.

16. The method of claim 10, wherein the acidic conditions are created by pumping CO₂ into the medium.

17. The method of claim 10, wherein the acidic conditions comprise a pH of 6 or less.

18. The method of claim 10, wherein the acidic condition comprises resuspending the cyanobacteria in 10 mM sodium acetate pH 5.2.

19. The method of claim 1, wherein the sucrose secreted exceeds 1 milligram per milliliter.

20. The method of claim 1, wherein the secreted sucrose is processed into concentrated molasses or dry sucrose crystals.

21. The method of claim 1, wherein the secreted sucrose is converted into a value added product selected from pharmaceuticals, vaccines, vitamins, industrial chemicals, proteins, pigments, fatty acids and their derivatives (such as polyhydroxybutyrate), acylglycerols (as precursors for biodiesel), and other secondary metabolites.

22. An isolated cyanobacterium comprising a portion of an exogenous bacterial cellulose operon sufficient to express bacterial cellulose, whereby the cyanobacterium is capable of producing secretable monosaccharides, disaccharides, oligosaccharides or polysaccharides comprising sucrose.
